(19) United States
(12) Patent Application Publication
(10) Pub. No.: US 2025/0064534 A1
(43) Pub. Date: Feb. 27, 2025

(54) MEDICAL SUPPORT METHOD, MEDICAL SUPPORT ROBOT DEVICE, AND COMPUTER PROGRAM

(71) Applicant: NATIONAL INSTITUTE OF ADVANCED INDUSTRIAL SCIENCE AND TECHNOLOGY, Tokyo (JP)

(72) Inventors: Ryousuke TSUMURA, Ibaraki (JP); Naotaka NITTA, Ibaraki (JP); Kiyoshi YOSHINAKA, Ibaraki (JP); Yoshihiko KOSEKI, Ibaraki (JP)

(73) Assignee: NATIONAL INSTITUTE OF ADVANCED INDUSTRIAL SCIENCE AND TECHNOLOGY, Tokyo (JP)

(21) Appl. No.: 18/719,225

(22) PCT Filed: Dec. 2, 2022
(86) PCT No.: PCT/JP2022/044587

§ 371 Date: Jun. 12, 2024

(30) Foreign Application Priority Data
Dec. 17, 2021 (JP) 2021-205070

(51) Int. Cl. A61B 34/30; A61B 7/02; A61B 8/00; A61B 34/20; G06T 7/00

(52) U.S. Cl. CPC A61B 34/30; G06T 7/0012; A61B 7/02; A61B 8/4218; A61B 2034/2065

(57) ABSTRACT

To provide a medical support method capable of making a medical instrument, such as an ultrasound probe and a stethoscope, autonomously contact with an appropriate position on a subject. A medical support method to be executed by a medical support robot device that performs medical practice to a subject using a medical instrument includes: a step (1) of imaging the subject at a plurality of imaging positions to acquire acquired three-dimensional point cloud information on the subject, two-dimensional image information, and imaging position coordinate information at each of the imaging positions, a step (2) of generating a single set of integrated three-dimensional point cloud information on the subject by using a plurality of sets of the acquired three-dimensional point cloud information and a plurality of sets of the imaging position coordinate information, a step (3) of determining a position of a specific site on the subject that is predetermined in a plurality of sets of the two-dimensional image information, and a step (4) of estimating a position of a diagnostic site on the subject that is subjected to the medical practice in the integrated three-dimensional point cloud information by using anatomical statistic information and the position of the specific site on the subject.

### TECHNICAL FIELD

The present disclosure relates to a medical support method to be executed by a medical support robot device.

### BACKGROUND ART

In the medical field, doctors perform medical practice such as examination of the body of a patient (a subject) as necessary. However, in many developed countries, the birthrate is declining and the population is aging, and so the proportion of doctors in the working generation is decreasing. Under these circumstances, it is expected that in near future, maintaining the quality of the existing medical services will become difficult. On the other hand, research and development in the field of robotics have remarkably progressed, and in recent years, robot devices have been used in various fields.

Therefore, also in the medical field, relatively safe treatments are increasingly entrusted to robot devices. For example, PTL 1 discloses a technique relating to improving the efficiency of ultrasound probe operation by a robot device. PTL 2 discloses a technique for making a medical instrument softly contact with a subject without using a force sensor.

### CITATION LIST

### Patent Literature

PTL 1: Japanese Patent Laid-Open No. 2020-157058
PTL 2: Japanese Patent Laid-Open No. 2014-100377

### SUMMARY OF INVENTION

### Technical Problem

However, PTL 1 and PTL 2 disclose only the techniques relating to the operation of an ultrasound probe or the like by the robot device. PTL 1 and PTL 2 do not disclose how the robot device autonomously determine to which of a subject a medical device, such as the ultrasound probe, needs to be applied when ultrasound diagnosis of the subject is performed by the robotic device.

Specifically, the conventional techniques partially automate scanning of the medical device, such as the ultrasonic probe, using the robotic device, although no useful proposal has been made for comprehensive automation of scanning that encompasses that the robot device determines a scanning position on a body surface of a subject in the state where the medical instrument is away from the subject and moves the medical instrument on the body surface.

In view of the above problems, an object of the present disclosure is to provide a medical support robot device capable of making a medical instrument, such as an ultrasound probe and a stethoscope, autonomously contact with an appropriate position on a subject.

### Solution to Problem

In order to solve the above problems, one aspect of the present disclosure relates to a medical support method to be executed by a medical support robot device that performs medical practice to a subject using a medical instrument, the medical support method including a step of imaging the subject at a plurality of imaging positions to acquire acquired three-dimensional point cloud information that is three-dimensional point cloud information on the subject, two-dimensional image information, and imaging position coordinate information at each of the imaging positions, a step of generating integrated three-dimensional point cloud information that is a single set of three-dimensional point cloud information on the subject by using a plurality of sets of the acquired three-dimensional point cloud information and a plurality of sets of the imaging position coordinate information, a step of determining a position of a specific site on the subject that is predetermined in a plurality of sets of the two-dimensional image information, and a step of estimating a position of a diagnostic site on the subject that is subjected to the medical practice in the single integrated three-dimensional point cloud information by using anatomical statistic information and the position of the specific site on the subject.

Another aspect of the present disclosure relates to a medical support robot device that performs medical practice to a subject using a medical instrument, the medical support robot device including an imaging sensor and a computer device, in which the imaging sensor images the subject at a plurality of imaging positions to acquire acquired three-dimensional point cloud information that is three-dimensional point cloud information on the subject, two-dimensional image information, and imaging position coordinate information at each of the imaging positions, and the computer device generates integrated three-dimensional point cloud information that is a single set of three-dimensional point cloud information on the subject by using a plurality of sets of the acquired three-dimensional point cloud information and a plurality of sets of the imaging position coordinate information, determines a position of a specific site on the subject that is predetermined in a plurality of sets of the two-dimensional image information, and estimates a position of a diagnostic site on the subject that is subjected to the medical practice in the single set of integrated three-dimensional point cloud information by using anatomical statistic information and the position of the specific site on the subject.

Still another aspect of the present disclosure relates to a computer program that causes the medical support robot device to execute one of the medical support methods.

### BRIEF DESCRIPTION OF DRAWINGS

FIG. 1 is a view showing an example of the configuration of a medical support robot device according to one embodiment of the present disclosure.

FIG. 2 is an explanatory view of coordinate transformation in the medical support robot device according to the one embodiment of the present disclosure.

FIG. 3 is a view showing an example of a processing flow for the medical support robot device according to the one embodiment of the present disclosure to autonomously perform auscultation to a subject.

FIG. 4 is a view showing an example of an anatomical map showing each placement position of the stethoscope on the body surface of the subject in two-dimensional space.

FIG. 5 is a view showing an example of procedures for the medical support robot device according to the one embodiment of the present disclosure to estimate the placement position.

FIG. 6 is a view showing an example of the configuration of an end effector of the medical support robot device according to the one embodiment of the present disclosure.

FIG. 7 is a view showing an example of the flowchart of processing to reconstruct a body surface shape in the medical support robot device according to the one embodiment of the present disclosure.

FIG. 8 is a view showing an example of the flowchart of the processing to reconstruct the body surface shape in the medical support robot device according to the one embodiment of the present disclosure.

FIG. 9 is a view showing an example of the flowchart of processing to estimate the placement position in the medical support robot device according to the one embodiment of the present disclosure.

FIG. 10 is a view showing an example of the flowchart of the processing to estimate the placement position in the medical support robot device according to the one embodiment of the present disclosure.

FIG. 11 is a view showing an example of the flowchart of constant load passive scanning in the medical support robot device according to the one embodiment of the present disclosure.

FIG. 12A is a view showing the positions of targets on a manikin in a multi-way registration experiment.

FIG. 12B is a view showing the positions of the targets on the manikin and four diagnostic sites in the multi-way registration experiment.

FIG. 13 is a view showing the results of multi-way registration with and without feedback of the position of a LiDAR camera.

FIG. 14 is a view showing the result of registration error that varies with the height of the LiDAR camera.

FIG. 15 is a view showing the result of positioning error for each estimated placement position.

FIG. 16A is a view showing the result of static contact force generated by a passive scanning mechanism in the example.

FIG. 16B is a view showing the result of dynamic contact force generated by the passive scanning mechanism in the example.

### DESCRIPTION OF EMBODIMENTS

Hereinbelow, embodiments of the present disclosure will be described in detail with reference to the drawings.

### (Configuration of Medical Support Robot Device)

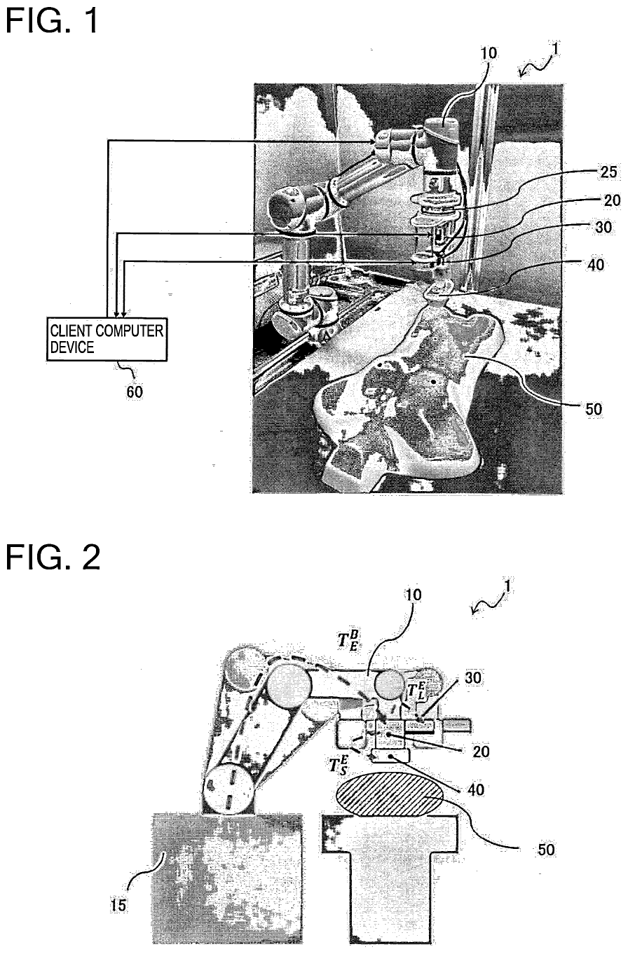

FIG. 1 is a view showing an example of the configuration of a medical support robot device according to the present embodiment. A medical support robot device 1 according to the present embodiment includes a robot arm 10, a constant load passive scanning mechanism (end effector) 20, and an RGB-D camera 30. The robot arm 10 is configured to be movable around a subject 50, which is a target of medical practice, such as the body of a patient (FIG. 1 is for illustrative purposes, and the subject 50 is substituted by a manikin). At the tip of the robot arm 10, the constant load passive scanning mechanism (end effector) 20 is provided integrally or detachably. The constant load passive scanning mechanism 20 is the mechanism that enables a medical instrument 40 to be held and enables the medical instrument 40 to be moved in the state of being pressed against the body surface of the subject 50 while a constant load is maintained. Here, the medical instrument may correspond to all kinds of instruments required for performing medical practice to the subject 50, such as ultrasound probes, and stethoscopes. More specific examples of the medical instrument usable in the medical support robot device 1 of the present embodiment may include electronic stethoscopes, such as electronic stethoscope (digital stethoscope) JPES-01 made by MITORIKA Co., Ltd. disclosed at https://www.milas.co.jp/product_stethoscope.html. Sounds collected when the electronic stethoscope is made to contact with the subject 50 can be transmitted as data to a computer device (such as a client computer device 60 described later). Similarly, other medical instruments are also usable if they can transmit sound and video data, acquired when the medical instruments are made to contact with the subject 50, to the computer device. The robot arm 10 can have the RGB-D camera 30 provided at its tip portion integrally or detachably. The RGB-D camera 30 is an example of an imaging sensor (ranging sensor) that can simultaneously acquire a color image (RGB information) and a distance image (distance information, depth information). The imaging sensor includes a projector that outputs laser light such as ultraviolet light, visible light, or infrared light, and a photoreceiver that receives reflected laser light that is reflected by a measurement target. Ranging methods for the imaging sensor include, for example, a time of flight (ToF) method and a pattern irradiation method. The ToF method is a ranging method for measuring the distance from an imaging sensor to a measurement target based on the time taken for the laser light, output from the projector, to be reflected by the measurement target and be received in the photoreceiver as reflection light. The pattern irradiation method is a ranging method in which a measurement target is irradiated with laser light with a specific pattern, and the distance from the image sensor to the measurement target is measured based on distortion of the pattern of reflected light coming from the measurement target. Note that the imaging sensor of the present embodiment is a laser imaging detection and ranging (LiDAR) camera. However, this is merely an example, and imaging sensors using other methods can be used.

The medical support robot device 1 may include the client computer device 60. The client computer device 60 is directly or indirectly connected to each of the robot arm 10, the constant load passive scanning mechanism (end effector) 20, and the RGB-D camera 30 of the medical support robot device 1 to transmit and receive data to and from these component members. For example, the client computer device 60 transmits a control signal to control each of the component members, and transmits data required to operate each of the component members. The client computer device 60 also receives data acquired or generated in each of the component members from the component members. The client computer device 60 can be implemented with a hardware configuration similar to that of general computer devices. The client computer device 60 may include, for example, a processor that can be implemented by a central processing unit (CPU) or an electronic circuit such as a microprocessor, a random access memory (RAM), a read only memory (ROM), a built-in hard disk device, a removable memory such as an external hard disk device, a CD, a DVD, a USB memory, a memory stick, and an SD card, an input-output user interface (display, keyboard, mouse, touch panel, speaker, microphone, LED, etc.), and a wired/wireless communication interface that can communicate with each component member of the medical support robot device 1 and other computer devices. In the client computer device 60, for example, a processor may read a computer program, which is stored in advance in a hard disk device, a ROM, or a removable memory, into a memory area such as a RAM and executes the program while reading necessary data from the hard disk device, the ROM, the removable memory, or the like as appropriate. This operation of the client computer device 60 implements various processing in the medical support robot device 1 described in detail below. Various types of data used in each processing described in the present embodiment are stored in a storage device or storage medium, such as a hard disk device, a RAM, and a removable memory, and are read into a memory area such as a RAM and used as necessary when the processor executes a computer program.

Some or all of the programs executed on the client computer device 60 according to the present embodiment can be stored in a computer-readable medium and be delivered, or can be downloaded via a wired or wireless communication network.

The medical support robot device 1 shown in FIG. 1 is a medical support robot device that was actually constructed by the inventors of this application by using: a six-axis collaborative 6-DOF cooperative robot arm (UR5e, Universal Robot. Denmark) as the robot arm 10; a LiDAR camera (Intel RealSense L515, Intel, USA) that acquires three-dimensional contours of the surface of the subject 50 as point cloud data, as the RGB-D camera 30; and a client computer device 60 (Dell Precision 5380, Dell, USA) to acquire point cloud data in synchronization with the robot arm 10. The medical support robot device 1 in this example is also constructed assuming auscultation using a stethoscope as an example. Since FIG. 1 shows the medical support robot device that is experimentally constructed, the subject 50 is a mannequin herein. Accordingly, since the nipples and navel set as landmarks of the subject 50 in this example are unclear on the manikin, these locations are marked with markers by the inventors as described later. The configuration shown in FIG. 1 is merely illustrative, and it is naturally understood that the configuration of the medical support robot device 1 according to the present embodiment is not limited to this. The same shall apply throughout this specification.

From the viewpoint of the safety of diagnosis of the subject 50, a constant load passive scanning mechanism using a spring is mounted on the end effector 20 of the robot arm 10 in order to grip the stethoscope (medical instrument) 40 adaptively to the body surface of the subject 50. At the base part of the constant load passive scanning mechanism (end effector) 20, a 6-axis force/torque sensor 25 (Axia-80-M20, ATI Industrial Automation, USA) is attached to measure contact force when the stethoscope 40 is placed on the body surface of the subject 50. The client computer device 60 that controls the medical support robot device 1 and an external server computer device (not shown) are directly connected to a network (speed: 1 gigabyte/second (GB/sec)), with TCP/IP used as a data transmission protocol. In the present embodiment, the LiDAR camera 30 is provided within the constant load passive scanning mechanism (end effector) 20, though the LiDAR camera 30 is not limited to this configuration. The LiDAR camera 30 is only required to be fixed so that its relative position to the robot arm 10 does not change, and may be installed at locations different from the location of the constant load passive scanning mechanism (end effector) 20.

The LiDAR camera 30 acquires information on the depth of the shape and the color of the subject 50 as point cloud data, and the point cloud data is used for coordinate registration (alignment) using the positional relation between the robot arm 10 and the subject 50. Since the LiDAR camera 30 is attached to the end effector 20 of the robot arm 10, the positional relation between the LiDAR camera 30 and the robot arm 10 remains kinematically fixed even when the robot arm 10 moves around the subject 50. Therefore, the positions of the acquired point cloud data on the subject 50 are linked with the coordinates of the robot arm 10. FIG. 2 is an explanatory view of coordinate transformation in the medical support robot device 1 in this example, which is constituted of the robot arm 10, the LiDAR camera 30, and the stethoscope (medical instrument) 40. A scanning point in coordinate space (P_{L}) of the LiDAR camera 30 is transformed to coordinate space (P_{B}) a base part 15 of the robot arm 10, and a contact point of the stethoscope 40 with the subject 50 in the coordinate space (P_{S}) is transformed into the coordinate space (P_{L}) of the LiDAR camera 30 (Formula (1)).

[Formula 1]

P_{B}=T_{E}^{B}T_{L}^{E}P_{L}

P_{S}=T_{S}^{E-1}T_{L}^{E}P_{L} (1)

### Here,

[Formula 2]

T_{E}^{B}

[Formula 3]

T_{S}^{E}

and

[Formula 4]

T_{L}^{E}

represent transformation from the base part 15 of the robot arm 10 to the end effector 20, transformation from the end effector 20 to the stethoscope 40, and transformation from the end effector 20 to the LiDAR camera 30, respectively.

[Formula 5]

T_{E}^{B}

can be determined by the controller of the robot arm 10.

[Formula 6]

T_{S}^{E}

and

[Formula 7]

T_{L}^{E}

can be calculated for each kinematic relation using a computer-aided design (CAD) model.

The robot arm 10 can be controlled by programming language URScript (Universal Robots A/S) used in the medical support robot device 1. The client computer device 60, which controls the medical support robot device 1, can transmit an URScript command to an external server computer device (not shown) via socket communication. The point cloud data was obtained using Intel RealSense SDK 2.0. A software system customized based on python programming using Visual Studio Code can synchronize control of the robot arm 10 with reading of the point cloud data.

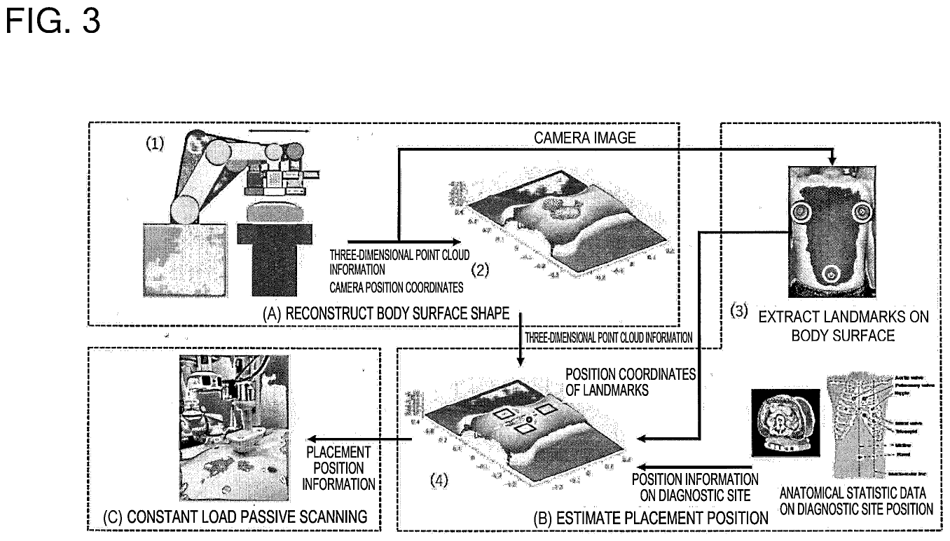

FIG. 3 is a view showing an example of the processing flow in which the medical support robot device 1 makes the stethoscope (medical instrument) 40 contact with the body surface of the subject 50 to perform autonomous auscultation. The processing flow mainly includes following component members (A) to (C):
(A) Reconstruction of body surface shape: acquiring point cloud data covering the entire chest of the subject 50 (processing (1)) and registration (alignment) of the acquired point cloud data to reconstruct the shape of the entire chest (processing (2)).
(B) Estimation of placement position: estimating the placement position of the stethoscope 40 (processing (4)) based on the reconstructed body shape, anatomical structure, and landmarks (markers) on the body surface of the subject 50 (processing (3)).
(C) Constant load passive scanning: placing the stethoscope 40 at the position estimated in (B) while maintaining a constant contact force.

Hereinafter, these three component members (A), (B) and (C) are described.

### (A) Reconstruction of Body Surface Shape (Registration of Point Cloud Data)

In order to accurately reconstruct the entire three-dimensional shape of the subject 50, several point cloud data sets are acquired by changing the position of the LiDAR camera 30, and registration (alignment) is performed. For this purpose, noisy data and partially overlapping data are handled. A common approach to this handling is to combine sampling-based rough alignment with iterative local search, such as iterative closest point (ICP) (see, for example, F. Pomerleau, F. Colas, R. Siegwart, and S. Magnenat, “Comparing ICP variants on real-world data sets: Open-source library and experimental protocol” Auton. Robots, vol. 34, no. 3, pp. 133-148, 2013). As an advanced registration algorithm, pairwise global registration, which is faster by one digit or more than general registration pipeline processing and more robust to noise, is widely used. This approach can also be employed in registration of a plurality of surfaces to acquire a model of large scenes or objects. This procedure is known as multi-way registration.

To further improve the accuracy of the registration, it is important to obtain the relative position of each data set. Assuming that point cloud data, which is paired with the position data for capturing the point cloud, is known, system coordinates of the captured data set (local coordinates in the medical support robot device 1, and this also applies to the following) can be transformed to global coordinates. As a result, it can be considered that registration errors can be minimized. An advantage of the medical support robot device developed by the inventors is that the position of the LiDAR camera 30 at the time of capturing each data set of the point cloud can accurately be obtained based on an encoder mounted on each joint of the robot arm 10.

Given a set of captured point cloud data {Pᵢ} on the body surface of the subject 50 following the multi-way registration, a set of position and posture information on each point cloud data set on global coordinates:

[Formula 8]

[symbol]

={Tᵢ}

needs to be estimated in global coordinates (see, for example, Q. Y. Zhou, J. Park, and V. Koltun, “Fast global registration”, Lect. Notes Artif. Intell., vol. 9906 LNCS, pp. 766-782, 2016). Because each captured point cloud data is on the coordinate space of the LiDAR camera 30, the coordinates can be transformed to the coordinate space of the robot arm 10 using the Formula (1) described above. An objective function of the multi-way registration can be written as follows (see, for example, Q. Y. Zhou, J. Park, and V. Koltun, “Fast global registration”. Lect. Notes Artif. Intell., vol. 9906 LNCS, pp. 766-782, 2016):

$$\begin{matrix}[ Formula 9 ] & \\ & ( 2 )\end{matrix}$$

$$E ( 𝕋 , 𝕃 ) = λ {\sum }_{i} {\sum }_{( p , q ) ∈ {𝒦}_{i}} {‖ {T}_{i} p - {T}_{i + 1} q ‖}^{2} + {\sum }_{i < j} ( {\sum }_{( p , q ) ∈ {𝒦}_{i , j}} {l}_{p , q} {‖ {T}_{i} p - {T}_{i + 1} q ‖}^{2} + {\sum }_{( p , q ) ∈ {𝒦}_{i , j}} Ψ ( {l}_{p , q} ) )$$

where

[Formula 10]

Kᵢⱼ

represents a set of candidates corresponding to the surface (Pᵢ, Qᵢ) of each pair. Here,

[Formula 11]

[symbol]

={l_{p,q}}

is a matrix of Black-Rangranjan duality (see, for example, M. J. Black and A. Rangarajan, “On the unification of line processes, outlier rejection, and robust statistics with applications in early vision”, Int. J. Compute. Vis., vol. 19, no. 1, pp. 57-91, 1996). Ψ(l_{p,q}) is set as follows:

[Formula 12]

Ψ(l_{p,q})=μ(\sqrt{l_{p,q}}−1) (3)

To solve a minimization problem, first,

[Formula 13]

E( [symbol] )

is minimized by:

[Formula 14]

[symbol]

To minimize the following:

[Formula 15]

E( [symbol] )

partial derivatives for each l_{p,q} are calculated as follows:

$$\begin{matrix}[ Formula 16 ] & \\ \frac{\partial E}{\partial {l}_{p , q}} = {‖ p - Tq ‖}^{2} + μ \frac{\sqrt{{l}_{p , q}} - 1}{\sqrt{{l}_{p , q}}} = 0 & ( 4 )\end{matrix}$$

Solving l_{p,q} with Equation (2) described above results in the following:

$$\begin{matrix}[ Formula 17 ] & \\ {l}_{p , q} = {( \frac{μ}{μ + {‖ p \to Tq ‖}^{2}} )}^{2} & ( 5 )\end{matrix}$$

Next, for all the poses:

[Formula 18]

[symbol]

[Formula 19]

E( [symbol] )

is minimized.

[Formula 20]

Tᵢ^{k}

is expressed as an i-th transformed pose calculated in the previous iteration. Tᵢ can be linearized by:

[Formula 21]

6-vector ξᵢ=(αᵢ, βᵢ, γᵢ, aᵢ, bᵢ,cᵢ)

as follows:

$$\begin{matrix}[ Formula 22 ] & \\ {T}_{i} \approx ( \begin{matrix}1 & - {γ}_{i} & {β}_{i} & {a}_{i ,} \\ {γ}_{i} & 1 & - {α}_{i} & {b}_{i} \\ - {β}_{i} & {α}_{i} & 1 & {c}_{i} \\ 0 & 0 & 0 & 1\end{matrix} ) {T}_{i}^{k} & ( 6 )\end{matrix}$$

$$When \{ {ξ}_{i} \} is matched with :$$

[Formula 23]

6| [symbol] |-vector

and the result is equal to =,

[Formula 24]

E( [symbol] )

is the objective function of least squares of ≡. Then, the objective function can be minimized by solving a linear system while updating Tᵢ as below:

[Formula 25]

Jᵣ^{T}JᵣΞ=−Jᵣ^{T}r (7)

where Jᵣ and r are a Jacobi matrix and a residual vector, respectively.

Based on the multi-way registration, registration pipeline processing in the present embodiment is as follows:

First, the LiDAR camera 30 attached to the end effector 20 of the robot arm 10 moves along several points in the state of being separated from the subject 50, and captures (images) the body surface of the subject 50. Then, the respective coordinates of the captured data sets are transformed to global coordinates based on the robot arm 10. Multi-way registration is executed using point cloud datasets expressed in global coordinates. Using transformation estimated for each dataset, each dataset is combined into a single dataset. Finally, filters are applied to the combined data to remove an inlier point cloud and an outlier point cloud.

### (B) Estimation of Placement Position

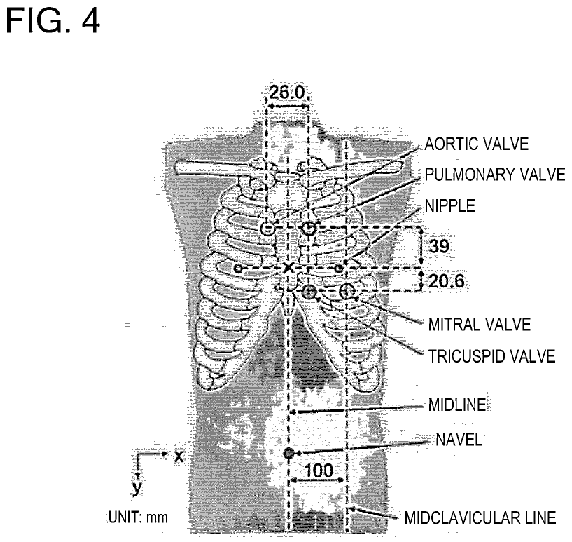

Auscultation is mainly performed to examine circulatory and respiratory systems. i.e., heart sounds and respiratory sounds. In the present embodiment, description is given of the examination of the circulatory system as an example. For examination of the circulatory system, a stethoscope is made to contact with four locations of the subject 50 to listen to the sounds of a tricuspid valve, a mitral valve, a pulmonary valve, and an aortic valve. As shown in FIG. 4, it is possible to listen to the sounds of the tricuspid valve, the mitral valve, the pulmonary valve, and the aortic valve generally at the left side of the lower sternum near the fifth intercostal space, above the apex of the heart in the fifth intercostal space on the left side of the midclavicular line (approximately 10 cm from the midline), above the inner end of the second intercostal space on the left, and above the inner end of the second intercostal space on the right. Therefore, in order to autonomously auscultate the sounds of these four locations, the medical support robot device 1 needs to recognize each of these positions.

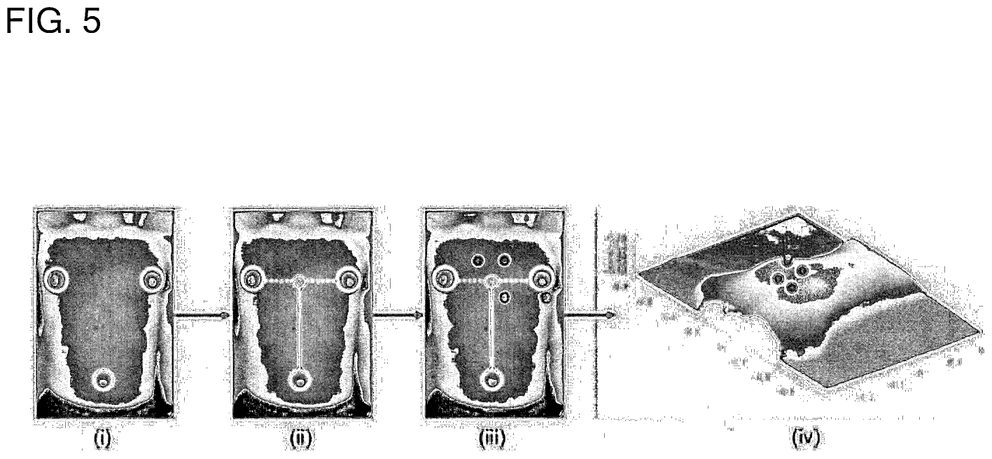

In the present embodiment, the positions of the nipples and the navel of the subject 50 are used to estimate each position on the body surface. The nipples and the navel are landmarks that are easily recognizable on the body surface and usable by the medical support robot device 1. The nipples are roughly positioned in the fourth intercostal space (FIG. 4). The positions can be the landmarks for finding each placement position of the stethoscope based on the anatomical relationship between each rib and its intercostal space. For example, as disclosed in J. A. Cook, S. S. Tholpady, A. Momeni, and M. W. Chu, “Predictors of internal mammary vessel diameter: A computed tomographic angiography-assisted anatomic analysis”. J. Plast. Reconstr. Aesthetic Surg., vol. 69, no. 10, pp. 1340-1348, 2016, the height of the intercostal space and ribs is measured using CT scan data. Based on the anatomical statistical data, the length between the fourth intercostal space (nipple positions) and the second intercostal space (aortic and pulmonary valve positions) along a cephalocaudal direction was 39 mm (millimeters), and an average length between the fourth intercostal space (nipple positions) and the fifth intercostal space (mitral valve and tricuspid valve positions) along the cephalocaudal direction was 20.6 mm. These lengths can be used as a reference height of each valve on a y-axis (cephalocaudal axis) of the system coordinates of the medical support robot device 1 (local coordinates in the medical support robot device 1). In addition, the position of the abdominal midline is necessary as a reference for estimating the width of each valve on an x-axis (lateral axis) of the system coordinate of the medical support robot device 1. The midline can be identified by connecting the midpoint between the left and right nipples to the navel with a straight line (FIG. 5). The midclavicular line on which the mitral valve is present is approximately 100 mm away from the midline along the x-axis (see, for example, R. A. Sayeed and G. E. Darling, “Surface Anatomy and Surface Landmarks for Thoracic Surgery”, Thorac. Surg. Clinic., vol. 17, no. 4, pp. 449-461, 2007). The stethoscope needs to be placed around the edge of the sternum, since other valves are positioned near the inner end of the intercostal space. The average width of the sternum of men is 25.99 mm (see, for example, J. S. Bruce. “Utility of the sternum to estimate sex and age”, Boston. Univ. Theses Diss., pp. 1-93, 2014, [Online] Available: https://hdl.handle.net/2144/15320). Next, it is assumed that the positions of the aortic valve and the pulmonary valves along the lateral axis are ±13 mm with the midline as a reference. In this way, an anatomical map indicating each placement position of the stethoscope on the body surface of the subject 50 in two-dimensional space can be created based on the reference position of each valve as shown in FIG. 4. It should be noted that the position of each examination location described above may be slightly deviated depending on the size of the subject 50 or the like. Therefore, the position of each examination location described above may have a certain allowance. For example, an actual value of the distance between the left and right nipples as the landmarks on the surface of the subject 50 may be compared with a statistical average value, and the length or other parameters used to identify each organ or each examination position described above may be enlarged or reduced in accordance with the difference between the actual value and the average value, so as to be used for estimation of the examination positions described later.

Thus, in the present embodiment, in order to estimate each placement position for placing the stethoscope, the positions of the nipples and the navel need to be identified from image information on the appearance of the subject 50 that is imaged with the LiDAR camera 30. By combining the identified positions of the nipples and the navel with the anatomical map of the body surface, each placement position of the stethoscope 40 for listening to the sounds of four valves can be estimated. The overall procedures for estimating the placement position is as follows (FIG. 5): (i) with use of a template matching method, the positions of the nipples and navel are extracted as two-dimensional pixel positions from a plurality of respective color images taken by the LiDAR camera 30; (ii) with use of the positions of the extracted nipples and the navel, the midpoint of the line between the left and right nipples is connected to the navel with a straight line to identify the position of the abdominal midline; (iii) with the identified midline position, each position of the diagnostic site on the two-dimensional pixel space of the color images can be estimated based on the anatomical map described above (FIG. 4); and (IV) the extracted two-dimensional pixel positions in the color images are projected into the three-dimensional coordinate space on the reconstructed body surface by transformation based on inherent camera parameters.

Although the examination of the circulatory system has been described in the foregoing, for the examination of the respiratory system, it is common to listen to the sounds of an upper lobe, a middle lobe, and a lower robe of the right and left lungs, and the sound of the trachea from the front chest and from the back with a stethoscope. These examination positions can be determined in the same way as described above. These examination positions may be more rough positions than those in the case of listening to the heart sounds.

### (C) Constant Load Passive Scanning

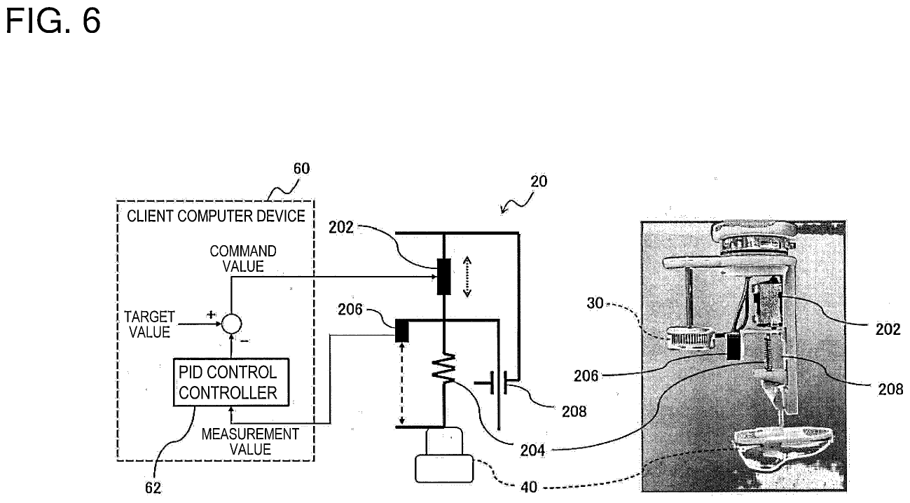

In order to safely place the stethoscope (medical instrument) 40, the inventors of this application have developed a novel end effector 20 having a spring-based passive scanning mechanism. FIG. 6 shows an example of the configuration of the developed end effector 20. The role of the passive scanning mechanism is to bring the stethoscope 40 into contact with the body surface of the subject 50 with a safe constant contact force, regardless of push-in displacement by the robot arm 10 when the stethoscope 40 is placed at the estimated placement position on the subject 50. The safe constant contact force may be a constant value predetermined by referring to the value of general pressure force when the doctor applies the stethoscope to the body of a patient. Here, there may be a certain error between the actual position of the stethoscope 40 and the placement position on the body surface because the body surface of the subject 50 is shifted due to such causes as respiratory motions of the patient. The end effector 20 according to the present embodiment can correct the error while maintaining the contact force within a certain range.

The end effector 20 shown in FIG. 6 is configured so as to include a linear servo actuator 202 (L1220PT, MigthyZap, Korea), a linear spring 204, an optical distance sensor 206 (ZX-LD100L. Omron, Japan), and a linear guide 208 (SSE2B6-70, Misumi, Japan). The linear servo actuator 202 moves up and down in a vertical direction with respect to the body surface of the subject 50 while maintaining a predetermined constant compression amount of the linear spring 204. In the present embodiment, a linear spring coefficient is 0.45 Newton/mm (N/mm), and two springs are inserted. The optical distance sensor 206 measures the amount of compression of the linear spring 204 in real time. The linear servo actuator 202 is controlled via an Arduino-based PID control controller 62 (IR-STS01, MigthyZap, Korea). The values measured with the optical distance sensor 206 are transferred to the client computer device 60 via a data acquisition (DAQ) tool (Analog Discovery 2, Digilent, USA). The software system of the client computer device 60, which is customized and designed based on Python programming with Visual Studio Code, synchronizes control of the linear servo actuator 202 with read processing of the measurement value data from the optical distance sensor 206. A proportional integral differential (PID) control scheme is used to control the position of the linear servo actuator 202 based on feedback from the optical distance sensor 206. More specifically, as shown in FIG. 6, the optical distance sensor 206 measures the distance from the optical distance sensor 206 to the body surface of the subject 50, and outputs the measurement value to the PID control controller 62. The measurement value is compared with a target value (a target distance between the optical distance sensor 206 and the body surface of the subject 50 to be maintained) to determine the compression amount of the linear spring 204 in consideration of the displacement of the body surface of the subject 50 due to such causes as respiratory motions of the patient, and the compression amount is output to the linear servo actuator 202 as a command value. The linear servo actuator 202 changes the compression state of the linear spring 204 based on the command value.

### (Flowchart)

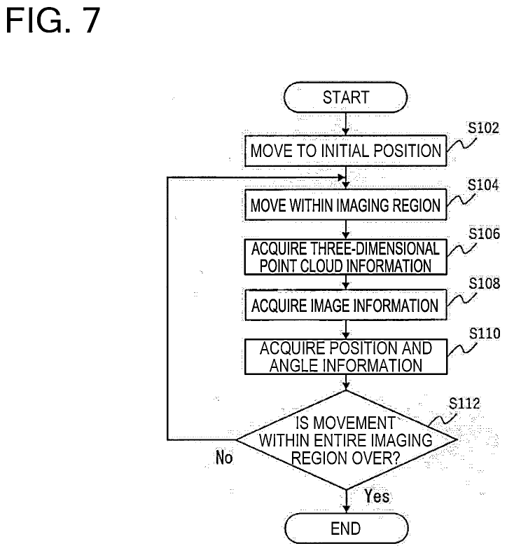

Hereinafter, description is given of an example of the processing performed by the medical support robot device 1 according to the present embodiment, with use of the flowcharts shown in FIGS. 7 to 11.

FIG. 7 shows an example of the flowchart of the processing ((1) in FIG. 3) in which the LiDAR camera 30 images the subject 50. First, the LiDAR camera 30 moves to a predetermined initial position (step S102). The LiDAR camera 30 acquires three-dimensional point cloud information (acquired three-dimensional point cloud information) at each imaging location (step S106), while moving within an imaging region (the entire chest of the subject 50 in the present embodiment) (step S104). At this time, the LiDAR camera 30 also acquires two-dimensional color image information at each imaging location and position and angle information (camera position coordinate information) on the LiDAR camera 30 at each imaging position (steps S108, S110). These processings are continued until the movement of the LiDAR camera 30 within the entire imaging region is over (step S112). These processings can be executed by controlling the robot arm 10 and the LiDAR camera 30 with control signals from the client computer device 60 (this also applies to each processing described below).

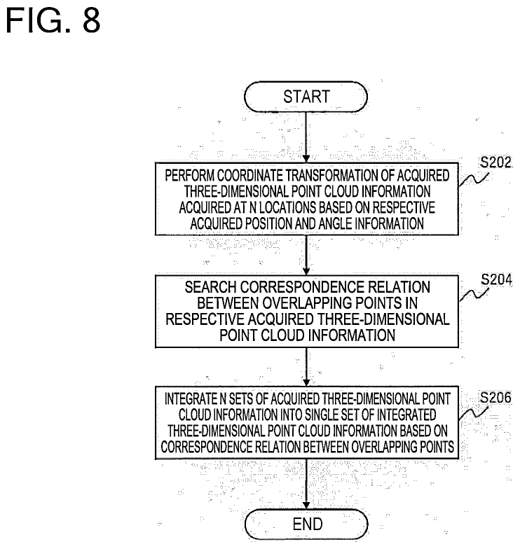

FIG. 8 is a view showing an example of the flowchart of the processing ((2) in FIG. 3) to reconstruct the body surface shape of the subject 50 by using a plurality of sets of acquired three-dimensional point cloud information and a plurality of sets of camera position coordinate information acquired in the processing of FIG. 7. The acquired three-dimensional point cloud information acquired at N locations (where N is an integer of two or more) are transformed from local coordinates to global coordinates based on the position and angle information (positional coordinate information) on the LiDAR camera 30 at each imaging position (step S202). Search for the correspondence relation between overlapping points in the respective acquired three-dimensional point cloud information is performed (step S204). The N sets of acquired three-dimensional point cloud information are integrated into a single set of three-dimensional point cloud information (integrated three-dimensional point cloud information) based on the correspondence relation between the searched overlapping points in step S204 (step S206).

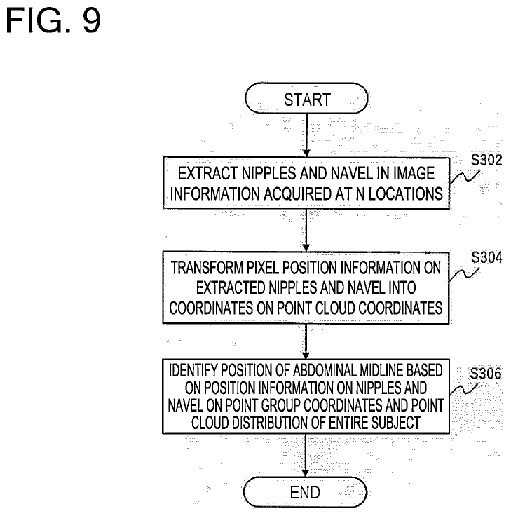

FIG. 9 is a view showing an example of the flowchart of the processing ((3) in FIG. 3) to extract the landmarks on the body surface of the subject 50. From each of the image information acquired at N locations, the nipples and the navel of the subject 50 are extracted by such method as template matching (step S302). Pixel position information on the extracted nipples and the navel are transformed into coordinates on the point cloud coordinates (step S304). The position of the abdominal midline is identified based on the position information (coordinates) of the nipples and the navel on the point cloud coordinates and on the point cloud distribution of the entire subject (step S306).

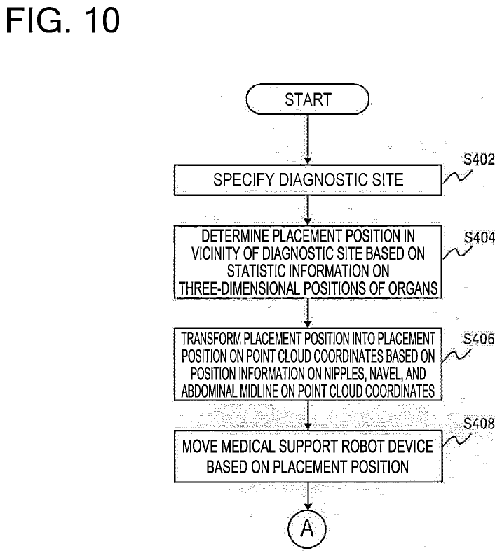

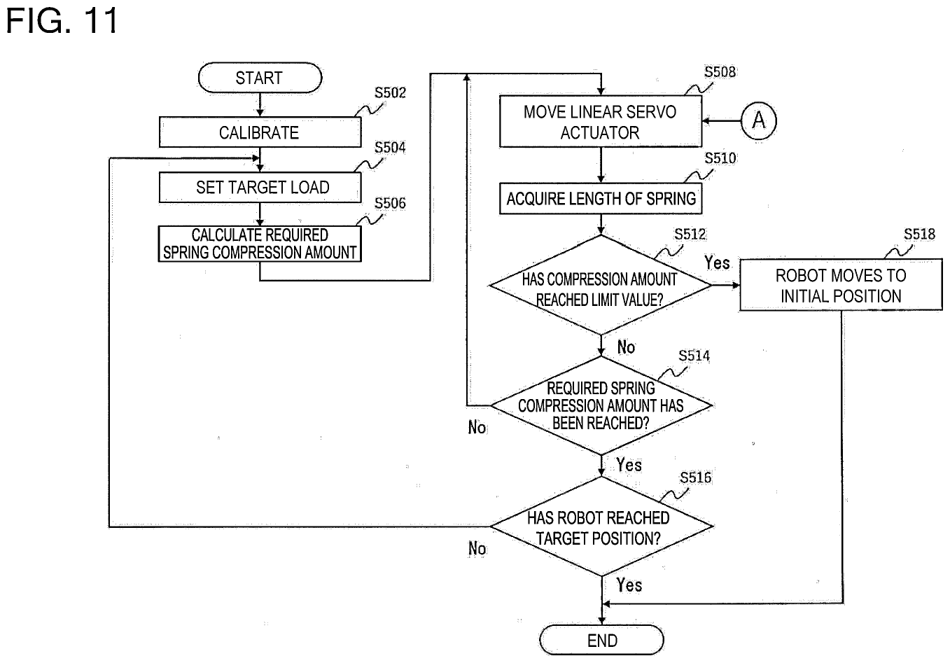

FIG. 10 is a view showing an example of the flowchart of the processing ((4) in FIG. 3) to estimate the placement position for placing the stethoscope 40. First, diagnostic sites are specified (step S402). In the present embodiment, the diagnostic sites are the sites to perform auscultation, which are four locations of the tricuspid valve, the mitral valve, the pulmonary valve, and the aortic valve. For example, a user (such as an operator) of the medical support robot device 1 may input these specifications using an input/output user interface, such as a keyboard, of the client computer device 60. The input content may be stored in a hard disk device, a RAM, or the like, and may be read from the hard disk device, the RAM, or the like, when step S402 is executed. Next, the placement position in the vicinity of a diagnostic site is determined based on the anatomical statistic information on the three-dimensional positions of the organs (step S404). The diagnostic site is transformed to the placement position on the point cloud coordinates by using as a reference the position information on the nipples, navel, and abdominal midline on the point cloud coordinates (step S406). The medical support robot device 1 moves based on the placement position of the diagnostic site on the point cloud coordinates that is transformed in step S406 (step S408). This is followed by step S508 in FIG. 11.

FIG. 11 is a view showing an example of the flowchart of constant load passive scanning ((C) in FIG. 3). First, in the medical support robot device 1, calibration of each component member is performed (step S502). The load (target load) used when the stethoscope (medical instrument) 40 is made to contact with the body surface of the subject 50 is set (step S504). For example, a user (such as an operator) of the medical support robot device 1 may input the setting using the input/output user interface, such as a keyboard, of the client computer device 60. The input content may be stored in a hard disk device, a RAM, or the like. Next, the client computer device 60 of the medical support robot device 1 calculates a required spring compression amount that is required for the target load set in step S504 (step S506). The medical support robot device 1 executes the processing of steps S502 to 506 as a preliminary preparation before the start of auscultation.

Subsequent to step S408 in FIG. 10, the robot arm 10 of the medical support robot device 1 operates based on the placement position of the diagnostic site on the point cloud coordinates that is transformed in step S406 in FIG. 10, and the linear servo actuator 202 of the constant load passive scanning mechanism 20, which is holding the stethoscope 40, moves to the placement position of the diagnosis site on the point cloud coordinates, where the stethoscope 40 is made to contact with the body surface of the subject 50 and is pressed with a predetermined contact force (step S508). The optical distance sensor 206 measures the length of the linear spring 204 (step S510). When the length of the linear spring 204 measured in step S510 reaches a limit value (step S512: Yes), no further processing can continue, and so the medical support robot device 1 moves to the initial position and ends the processing (step S518). In this case, processing such as changing an initial value of the contact force of the stethoscope 40 may be performed in step S508, and then auscultation processing may be re-executed.

When the length of the linear spring 204 measured in step S510 does not reach the limit value (step S512: No), the client computer device 60 of the medical support robot device 1 determines whether the length of the linear spring 204 measured in step S510 reaches the required spring compression amount calculated in step S506 (step S514). When the length does not reach the required spring compression amount (step S514: No), the processing returns to step S508 due to the lack of contact force, and the linear servo actuator 202 is operated so as to increase the compression amount of the linear spring 204. When the client computer device 60 of the medical support robot device 1 determines that the length of the linear spring 204 reaches the required spring compression amount in step S514 (step S514: Yes), auscultation of the diagnostic site is performed with the stethoscope 40. When, for example, the medical instrument 40 is an ultrasound probe, diagnostic images detected by the ultrasound probe are output to the client computer device 60 at any time while the ultrasound probe is pressed and moved in a specific region on the body surface of the subject 50. A doctor can view the diagnostic images on the client computer device 60 or a remote computer device to perform diagnosis. The client computer device 60 also determines the compression amount of the linear spring 204 in consideration of the displacement of the body surface of the subject 50 due to such causes as respiratory motions of the patient, and determines whether the stethoscope 40 has accurately reached the target position (step S516). When the stethoscope 40 does not accurately reach the target position, the processing may return to step S508, where the position of the constant load passive scanning mechanism 20 holding the stethoscope 40 may be adjusted.

### EXPERIMENTAL EXAMPLES

In order to verify the proof of concept of the medical support robot device according to the present embodiment, which enables autonomous positioning of the stethoscope 40 based on the information on the appearance of the subject 50 while ensuring the safety of the patient, the inventors conducted three main types of experiments. In all the experiments, a white male torso mannequin was used as the subject 50. Since the manikin does not have nipples and navel, markers were placed at their respective positions.

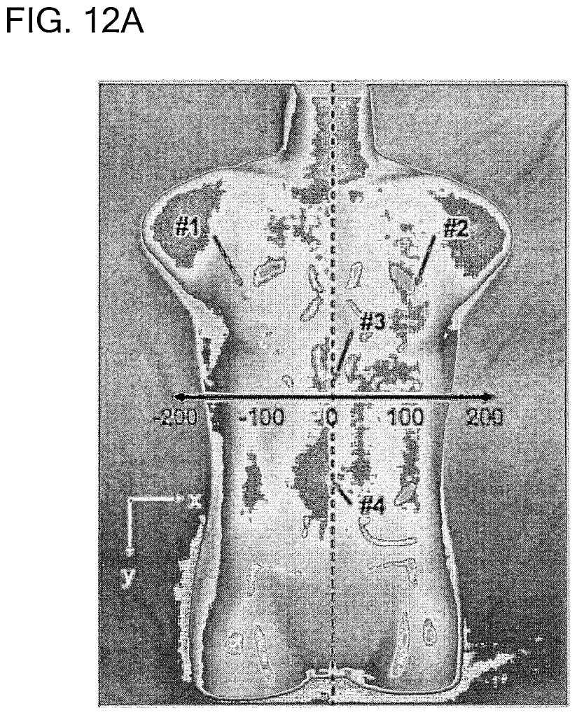

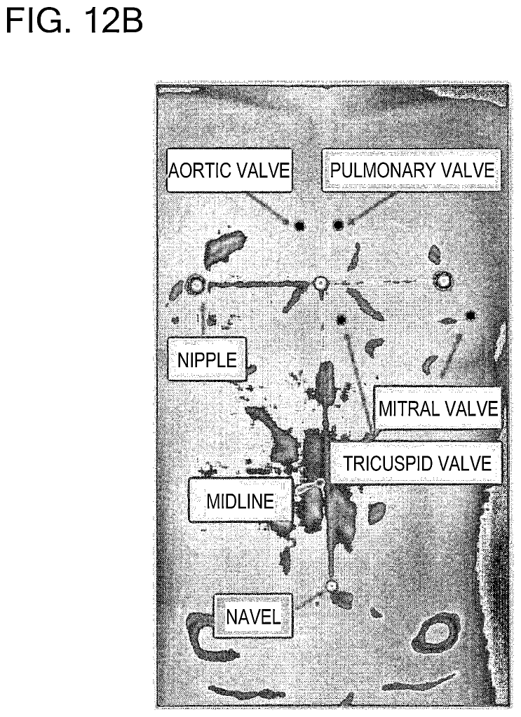

First, the accuracy of the reconstructed body surface using the RGB-D camera 30, described in “(A) Reconstruction of body surface shape (point cloud data registration)”, was evaluated. FIG. 12A is a view showing the positions of the targets on a manikin in a multi-way registration experiment. FIG. 12B is a view showing the position of the targets on the manikin and four diagnostic sites in this experiment. For multi-way registration, point cloud data was acquired (imaged with the LiDAR camera 30) at five positions, i.e., at the positions of −200 mm, −100 mm, 0 mm, 100 mm, and 200 mm along the x-axis with the midline of the subject 50 as an origin, as shown in FIG. 12A. Furthermore, the performance of the multi-way registration with the feedback from the position of the LiDAR camera 30 (Equation (1)) was compared with the performance without the feedback. The feedback here refers to the processing of coordinate transformation of each point cloud data to the system coordinates of the medical support robot device 1 (local coordinates in the medical support robot device 1) based on the position information (Equation (1)) on the LiDAR camera 30. Since the accuracy of the point clouds depends on the distance between the LiDAR camera 30 and the subject, point cloud data was obtained by varying the distance of the LiDAR camera 30 from the subject 50 in three stages (250 mm, 300 mm, and 350 mm from the upper end of the chest of the subject 50). To evaluate the accuracy of the multi-way registration, the robot arm 10 was moved to the four targets on the subject 50, that is, (moved) to the left and right nipples, the cardiac space, and the navel (FIG. 12A) based on the reconstructed body surface data, and the distance between the tip of the end effector 20 and each target was measured in three-dimensional coordinate space. The position of each target was manually indicated on the body surface data reconstructed in this experiment.

Moreover, to eliminate other factors (for example, errors due to assembly of mechanical parts) without using the registration methods, a jig was attached to the robot arm 10 and the center of the end effector 20 was identified (FIG. 12A). Twelve tests were performed for each condition.

Next, the accuracy of the method of estimating the placement position for placing the stethoscope 40, described in “(B) Estimation of placement position”, was evaluated. With use of the reconstructed body surface data (the height of the LiDAR camera 30:300 mm) used in the above experiment, four placement positions, namely, the positions of the tricuspid valve, the mitral valve, the pulmonary valve, and the aortic valves, were estimated. Based on the anatomical map shown in FIG. 4, markers were added for the respective placement positions on the manikin. The position of the manikin was slightly varied in 12 random patterns.

In addition, the safety of the end effector 20 having the passive scanning mechanism described in “(C) Constant load passive scanning” was evaluated by measuring both static and dynamic contact forces. The term “static contact force” refers to the contact force measured with the medical instrument (stethoscope) 40 in contact with the body surface in a stationary state. The term “dynamic contact force” refers to the contact force generated during the operation of bringing the medical instrument (stethoscope) 40 in air into contact with the body surface. In this experiment, the static contact force was measured after the stethoscope 40 was pressed 5 mm against the ground. Three types of contact forces (5N, 10N, and 15N) were set. For each condition, 12 trials were performed. The dynamic contact force was measured when the stethoscope 40 was moved from air to the body surface of the mannequin. The initial position of the stethoscope 40 was 5 mm from the body surface of the subject 50 along the z-axis (a perpendicular direction of the subject 50 (manikin) lying in supine posture). In this state, the stethoscope 40 was moved 10 mm along the z-axis and pressed 5 mm against the body surface of the subject 50. The target contact force in this experiment was set to 5N.

### Results of Experiments

### (1) Reconstruction of Body Surface Shape

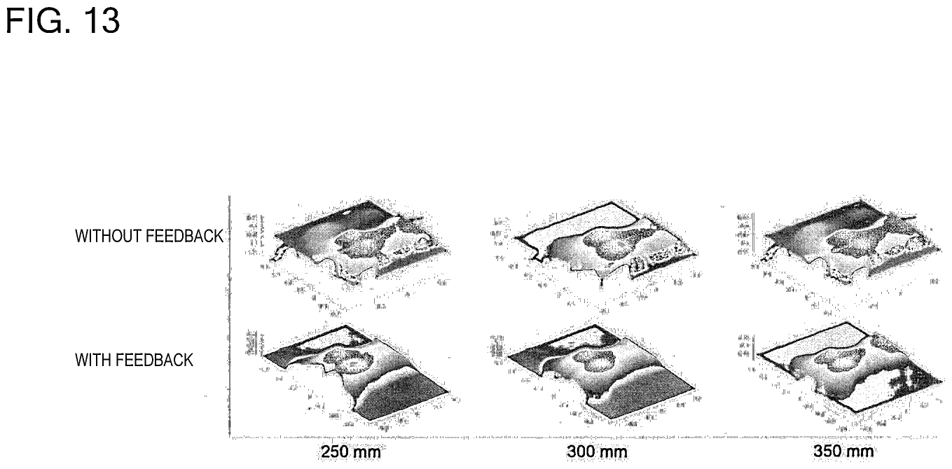

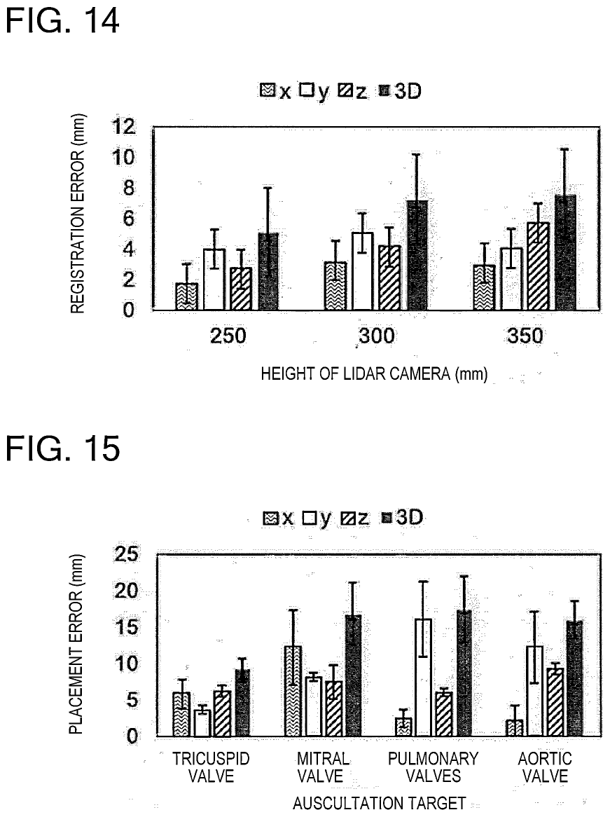

FIG. 13 shows the result of the multi-way registration with and without feedback of the position of the LiDAR camera 30. The upper row shows the estimation result for each height without position feedback. The lower row shows the estimation result for each height with position feedback. The results shown in FIG. 13 indicate that in all the heights, the reconstruction with position feedback can provide more accurate chest shape of the subject 50 than the reconstruction without position feedback from the LiDAR camera 30. FIG. 14 is a view showing the result of registration error that varies with the height of the LiDAR camera 30. The results shown in FIG. 14 indicate that as the distance between the LiDAR camera 30 and the target is shorter, the errors are reduced more. Whether there is a significant difference in accuracy depending on the height of the LiDAR camera 30 was determined using two-tailed student's t-test having 90% confidence intervals. There was a significant difference in error of three-dimensional spatial coordinates between the heights of 250 mm and 350 mm of the LiDAR camera 30 (p<0.05).

### (2) Estimation of Placement Position

FIG. 15 is a view showing the result of positioning error for each estimated placement position. The error of the tricuspid valve was smaller than the error of other valves. The two-tailed student's t-test with 90% confidence intervals was also used for positioning accuracy dependent on the position of the target valve. There was a significant difference in error of three-dimensional spatial coordinates between the tricuspid valve and the other valves (p<0.01).

### (3) Contact Force of Constant Load Passive Scanning Mechanism

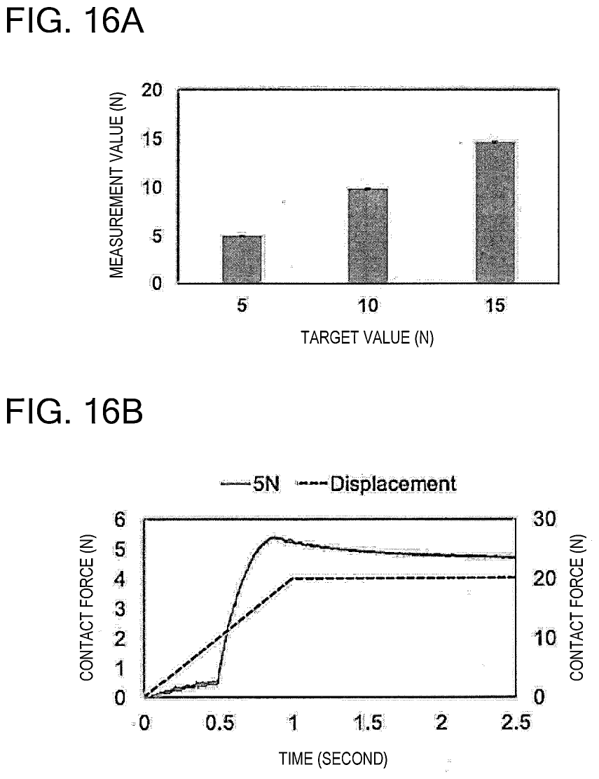

FIG. 16A is a view showing the result of static contact force generated by the passive scanning mechanism in this example. FIG. 16B is a view showing the result of dynamic contact force generated by the passive scanning mechanism in this example. The result of the static contact force showed that the generated contact force (vertical axis) was accurately achieved with respect to the target force (horizontal axis) under all the conditions. Based on the result of the dynamic contact force, the time series measurement value increased (0.5 seconds) when in contact with the body surface of the subject 50, and slightly exceeded the target value. However, the measurement value was immediately adjusted to meet the target value. The maximum contact force measured was 5.36N, which was 7.2% error of the target value.

According to the medical support robot device according to the present embodiment as described in the foregoing, when medical practice is performed to a subject using a medical instrument, the medical support robot device can automatically determine the position to contact the medical instrument without using a manual operation. After determination, it is also possible to move the medical instrument on the body surface while maintaining a predetermined contact force. Such a function can achieve full automatic operation of the medical support robot device which could only be operated in a semi-automatic mode in the past.

In addition, the full automation of the medical support robot device makes it possible to maintain the quality of medical practice even in countries with declining birthrates and aging populations. Conventional auscultations and ultrasound examinations are important examinations in the clinical settings to detect abnormal clinical signs. However, conventional auscultations and ultrasound examinations require doctors to physically contact with the patients. As a result, doctors were exposed to infectious diseases through the patients, and were required to contact the stethoscope or ultrasound probe with an optimal contact force with the subjects, which required the doctors to have specialized skills. Since the medical support robot according to the present embodiment is fully automated, remote medical care also becomes available, which is beneficial in terms of protecting doctors against infection, and also allow patients to receive high-quality medical services even in areas where medical resources are scarce.

Although one embodiment of the present disclosure has been described in the foregoing, the present disclosure is not limited to the embodiment disclosed, and it goes without saying that the present disclosure can be implemented in various forms without departing from the scope of the technical idea.

For example, in the embodiment described above, the nipples and the navel are used as landmarks on the body surface of the subject 50. However, any site having notable appearance in the human body can be used. For example, joints such as the shoulder, the clavicle, or the pelvis are easier to identify from the outside, so that it is expected that each joint part can be used as a landmark. For estimating the position information on each joint of the subject 50, publicly-known software (for example. “OpenPose” that is an open-source software) can be used.

In the embodiment disclosed, the position of the abdominal midline is determined from the position of the nipples and the navel that are landmarks, and the position of each diagnostic site on the point cloud coordinates are estimated using the anatomical positional relationship between the abdominal midline and each diagnostic site. However, depending on the positional relationship between the landmarks and the diagnostic sites, it is of course possible to estimate the position of the diagnostic sites directly from the position of the landmarks.

In the above embodiment, the case of performing auscultation of the subject 50 using a stethoscope has been described in detail, though this is merely an example. For example, the medical instrument may be another medical instrument, such as an ultrasound probe used for ultrasound examination. For example, the present disclosure is also applicable to the case of moving a medical instrument, such as an ultrasound probe, with a predetermined contact force while pressing the medical instrument against a specific region on the body surface of the subject 50.

Furthermore, the scope of the present disclosure is not limited to the illustrated and described exemplary embodiments but includes all embodiments that provide the effects equivalent to the effects that are intended to be attained by the present disclosure. Furthermore, the scope of the present disclosure is not limited to a combination of features of the present disclosure defined by each claim, and may be defined by any desired combination of specific features among all of the respective disclosed features.

### REFERENCE SIGNS LIST

1 . . . . Medical support robot device
10 . . . . Robot arm
15 . . . . Base part
20 . . . . Constant load passive scanning mechanism (end effector)
202 . . . . Linear Servo Actuator
206 . . . . Distance sensor
206 . . . . Light distance sensor
208 . . . . Linear guide
25 . . . . Force/torque sensor
30 . . . . RGB-D camera (LiDAR camera)
40 . . . . Medical instrument (stethoscope)
50 . . . . Subject
60 . . . . Client computer device
62 . . . . PID control controller

1. A medical support method to be executed by a medical support robot device that performs medical practice to a subject using a medical instrument, the medical support method comprising:
a step of imaging the subject at a plurality of imaging positions to acquire acquired three-dimensional point cloud information that is three-dimensional point cloud information on the subject, two-dimensional image information, and imaging position coordinate information at each of the imaging positions;
a step of generating integrated three-dimensional point cloud information that is a single set of three-dimensional point cloud information on the subject and that indicates a shape of a surface of a part of the subject by using a plurality of sets of the acquired three-dimensional point cloud information and a plurality of sets of the imaging position coordinate information;
a step of determining a position of a specific site on the subject that is predetermined in a plurality of sets of the two-dimensional image information; and
a step of estimating a position of a diagnostic site on the subject that is subjected to the medical practice in the single set of integrated three-dimensional point cloud information by using anatomical statistic information and the position of the specific site on the subject.

2. The medical support method according to claim 1, wherein in the step of estimating, the position of the diagnostic site is estimated by using the anatomical statistic information and a position of another site that is determined from the position of the specific site on the subject.

3. The medical support method according to claim 1, wherein
the plurality of sets of the imaging position coordinate information are local coordinate information on the medical support robot device, and
the step of generating the single set of integrated three-dimensional point cloud information includes
a step of transforming, based on local coordinate information on the imaging positions at a time when each of the plurality of sets of the acquired three-dimensional point cloud information is acquired, the local coordinate information on each of the plurality of sets of the acquired three-dimensional point cloud information to global coordinates,
a step of searching correspondence relation between overlapping points in the plurality of sets of the acquired three-dimensional point cloud information, and
a step of integrating the plurality of sets of the acquired three-dimensional point cloud information into the single set of integrated three-dimensional point cloud information based on the correspondence relation between the searched overlapping points.

4. The medical support method according to claim 1, wherein the specific site on the subject includes any one of left and right nipples and navel of the subject, and each joint of the subject.

5. The medical support method according to claim 1, further comprising:
a step of acquiring a target load to be applied when the medical instrument is made to contact with the diagnostic site; and
a step of making the medical instrument contact with the estimated position of the diagnostic site with the target load.

6. The medical support method according to claim 5, wherein the medical support robot device includes a spring that makes the medical instrument contact with the diagnostic site with the target load, and
a compression amount of the spring required for the target load is calculated based on the acquired target load.

7. The medical support method according to claim 6, further comprising:
a step of measuring the compression amount of the spring while the medical instrument is made to contact with the diagnostic site; and
a step of adjusting the compression amount of the spring while the medical instrument is made to contact with the diagnostic site by using the compression amount of the spring required for the calculated target load and the compression amount of the spring measured while the medical instrument is made to contact with the diagnostic site.

8. A medical support robot device that performs medical practice to a subject using a medical instrument, the medical support robot device comprising an imaging sensor and a computer device, wherein
the imaging sensor
images the subject at a plurality of imaging positions to acquire acquired three-dimensional point cloud information that is three-dimensional point cloud information on the subject, two-dimensional image information, and imaging position coordinate information at each of the imaging positions, and
the computer device
generates integrated three-dimensional point cloud information that is a single set of three-dimensional point cloud information on the subject and that indicates a shape of a surface of a part of the subject by using a plurality of sets of the acquired three-dimensional point cloud information and a plurality of sets of the imaging position coordinate information,
determines a position of a specific site on the subject that is predetermined in a plurality of sets of the two-dimensional image information, and
estimate a position of a diagnostic site on the subject that is subjected to the medical practice in the single integrated three-dimensional point cloud information by using anatomical statistic information and the position of the specific site on the subject.

9. The medical support robot device according to claim 8, wherein
a plurality of sets of the imaging position coordinate information are local coordinate information on the medical support robot device, and
generating the single set of the integrated three-dimensional point cloud information includes
transforming, based on local coordinate information on the imaging positions at a time when each of the plurality of sets of the acquired three-dimensional point cloud information is acquired, the local coordinate information on each of the plurality of sets of the acquired three-dimensional point cloud information to global coordinates,
searching correspondence relation between overlapping points in the plurality of sets of the acquired three-dimensional point cloud information, and
integrating the plurality of sets of the acquired three-dimensional point cloud information into the single set of integrated three-dimensional point cloud information based on the correspondence relation between the searched overlapping points.

10. The medical support robot device according to claim 8, wherein
the computer device
acquires a target load to be applied when the medical instrument is made to contact with the diagnostic site, and
outputs a control signal for making the medical instrument contact with the estimated position of the diagnostic site with the target load.

11. The medical support robot device according to claim 10, wherein
the medical support robot device includes a spring that makes the medical instrument contact with the diagnostic site with the target load, and
the computer device calculates, based on the acquired target load, a compression amount of the spring required for the target load.

12. The medical support robot device according to claim 11, wherein
the computer device
acquires the compression amount of the spring while the medical instrument is made to contact with the diagnostic site, and
calculates the compression amount of the spring while the medical instrument is made to contact with the diagnostic site by using the compression amount of the spring required for the calculated target load and the compression amount of the spring measured while the medical instrument is made to contact with the diagnostic site.

13. A non-transitory computer-readable medium storing a computer program that causes a medical support robot device to execute the medical support method according to claim 1.
